(19) United States
(12) Patent Application Publication
(10) Pub. No.: US 2023/0302023 A1
(43) Pub. Date: Sep. 28, 2023

(54) ANTI-ACNE SKIN CARE COMPOSITIONS

(71) Applicant: Edgewell Personal Care Brands, LLC, Chesterfield, MO (US)

(72) Inventor: Begona Stevenson, Coppell, TX (US)

(21) Appl. No.: 18/191,231

(22) Filed: Mar. 28, 2023

Related U.S. Application Data
(60) Provisional application No. 63/324,276, filed on Mar. 28, 2022.

(51) Int. Cl. A61K 31/60; A61K 47/34; A61K 36/074; A61K 36/886; A61K 36/9066; A61K 36/481; A61K 36/82; A61K 47/12; A61P 17/10

(52) U.S. Cl. CPC A61K 31/60; A61K 47/34; A61K 36/074; A61K 36/886; A61K 36/9066; A61K 36/481; A61K 36/82; A61K 47/12; A61P 17/10

(57) ABSTRACT

A skin care composition includes an anti-acne active ingredient such as a sulfur ingredient, salicylic acid, or combinations thereof. The skin care composition further includes an odor neutralizer, an alpha hydroxy acid, or combinations thereof. In one example, the anti-acne active ingredient includes sulfur and the odor neutralizer includes a polymer to reduce and/or eliminate the odor caused by the sulfur. Additionally or alternatively, the alpha hydroxy acid includes a mixture comprising lactic acid, glycolic acid, malic acid, mandelic acid, tartaric acid, and citric acid to provide skin care benefits such as exfoliation with improved moisturization. A method to treat and/or prevent acne may include applying to facial skin one or more of a cleansing skin care composition, a treatment skin care composition, or a moisturizing skin care composition to minimize acne, irritation, dryness, odor, and the like.

### CROSS-REFERENCE TO RELATED APPLICATIONS

This application claims priority to U.S. Provisional Patent Application No. 63/324,276, filed on Mar. 28, 2022, the content of which is incorporated herein by reference in its entirety.

### TECHNICAL FIELD

Embodiments relate to anti-acne skin care compositions including an anti-acne active ingredient and an ingredient selected from an odor neutralizer and/or an alpha hydroxy acid.

### BACKGROUND

Skin care compositions may include a variety of active ingredients to prevent or treat acne. Sulfur, for example, is highly effective. As a result of the unpleasant odor of sulfur, however, individuals are reluctant to utilize such skin care compositions on the body. Various technologies have therefore been proposed to mask or neutralize the malodor of sulfur including using perfumes, zinc salts of ricinoleic acid, mineral powders (bentonite, hectorite, smectite, kaolin, saponite, carbonate), and the like. Such skin care compositions may not be entirely satisfactory or may cause unintended consequences (e.g., irritation, dryness, etc.). Polymers, such as sodium zinc polyitaconate, have also been proposed in antiperspirant compositions to mask the malodor from body surfaces (e.g., under the arms). Given the intended use of such ingredients and given that the top layer of facial skin (stratum corneum) is more delicate than the rest of the skin on the body, incorporation of such polymers into topically applied facial skin care compositions has traditionally been limited.

Meanwhile, ingredients added to facial skin care compositions for certain skin benefits can nonetheless cause unintended consequences. In one example, exfoliants may cause the skin to become more sensitive to irritation triggered by other topically applied ingredients such as polymers, moisturizers, sunscreens, fragrances, surfactants, and the like. Exfoliants may also increase the skin's sensitivity to environmental conditions such as sunlight, wind, cold temperature, and dry air, or may exacerbate irritation from a pre-existing skin disease. In a further example, skin-conditioning agents may clog pores and cause breakouts. Thus, consumers are left to avoid such ingredients or assume the unintended consequences.

There is considerable room for improvement to provide a skin care composition, having an anti-acne agent and one or more of an odor neutralizer or an alpha hydroxy acid, with an acceptable skin benefit profile (e.g., anti-acne, low odor, good moisturization, etc.).

### SUMMARY

Embodiments relate to a skin care composition including an anti-acne active ingredient and an ingredient selected from the group consisting of an odor neutralizer, an alpha hydroxy acid (AHA), or combinations thereof. The anti-acne active ingredient may be selected from the group consisting of salicylic acid, a sulfur ingredient, or combinations thereof. The sulfur ingredient may be selected from the group consisting of elemental sulfur, a sulfide, a sulfite, a mercaptan, or combinations thereof. Thus, the sulfur ingredient may include sulfur powder. The anti-acne active ingredient may be present in an amount from about 0.001 wt. % to about 20.0 wt. %, including every number and/or fraction therebetween, based on the total weight of the skin care composition.

The odor neutralizer may be selected from a perfume, a metal oxide, a mineral powder, a polymer, or combinations thereof. The odor neutralizer may be free of a perfume, a metal oxide, and a mineral powder. Additionally or alternatively, the odor neutralizer may be selected from a polymer in salt form. For example, the polymer may be selected from a copolymer having at least one zinc counterion. The polymer may be in a mixed salt form of sodium ions and zinc ions. Additionally or alternatively, the polymer may be selected from a copolymer of taurides and itaconic acid. The odor neutralizer may be present in an amount from about 0.001 wt. % to about 7.5 wt. %, including every number and/or fraction therebetween, based on the total weight of the skin care composition.

The AHA may be selected from an alkyl AHA, an aralkyl AHA, a polycarboxyl AHA, or combinations thereof. The AHA may consist of an alkyl AHA, an aralkyl AHA, and a polycarboxyl AHA. In one example, the AHA may consist of lactic acid, glycolic acid, mandelic acid, malic acid, tartaric acid, and citric acid. The AHA may be present in an amount from about 0.1 wt. % to about 15.0 wt. %, including every number and/or fraction therebetween, based on the total weight of the skin care composition.

The skin care composition may include one or more other ingredients. In one example, the one or more other ingredients may include at least one auxiliary skin benefit agent. The auxiliary skin benefit agent may be present in an amount from about 0.001 wt. % to about 10.0 wt. %, based on the total weight of the skin care composition. For example, Curcuma longa (turmeric) root extract, Ganoderma lucidum (mushroom) extract, Astragalus membranaceus root extract, Aloe barbadensis, Camellia sinensis leaf extract, hydroxycinnamic acid, rutin, sodium hyaluronate, lysine, 10-hycroxydecanoic acid, and/or bisabolol may be present in an amount of 0.002 wt. %, 0.005 wt. %, 0.01 wt. %, 0.015 wt. %, 0.02 wt. %, 0.03 wt. %, 0.04 wt. %, 0.05 wt. %, 0.07 wt. %, 0.08 wt. %, 0.1 wt. %, 0.15 wt. %, 0.2 wt. %, 0.5 wt. %, 0.70 wt. %, 1.0 wt. %, 1.25 wt. %, 1.5 wt. %, 1.75 wt. %, 2.0 wt. %, 2.5 wt. %, 3.0 wt. %, 3.5 wt. %, 5.0 wt. %, 7.5 wt. %, 10.0 wt. %, 12.0 wt. %, including every number and/or fraction therebetween, based on the total weight of the skin care composition.

Embodiments relate to a consumer-packaged product comprising a container housing the skin care composition of any of the preceding embodiments. Embodiments may include using various packaged formats such as, without limitation, pump containers, spray containers, mist containers, tubes, jars, boxes, and the like. The container may be made of any suitable material such as, without limitation, plastic, compostable material, biodegradable material, metal, or combinations thereof. Moreover, the skin care composition may be in any cosmetically acceptable form such as, without limitation, an emulsion, a gel, a lotion, a cream, a spray, a mist, a stick, a mousse, a foam, an ointment, a milk, a base, a concentrate, or combinations thereof. Thus, embodiments may include making and packaging the skin care compositions using any traditional process.

Embodiments relate to a method to provide an anti-acne skin benefit to an individual including contacting skin of the individual with the skin care composition of any of the preceding embodiments. Thus, the method may include applying the skin care composition from a container onto the face, onto the neck, and so on. In one example, the method may include sequentially contacting the skin with two or more of a cleansing skin care composition, a treatment skin care composition, or a moisturizing skin care composition. The method may also provide an additional benefit such as relatively less odor from an ingredient present in the skin care composition and/or exfoliation of the skin with moisturization when compared to a condition of the skin without the use of the skin care composition or relative to a condition of the skin with the use of a different anti-acne skin care composition.

### DETAILED DESCRIPTION

Embodiments can comprise, consist of, and consist essentially of the features and/or steps described herein, as well as any of the additional or optional ingredients, components, steps, or features described herein or would otherwise be appreciated by one of skill in the art. It is to be understood that all concentrations disclosed herein are by weight percent (wt. %.) based on a total weight of the composition unless otherwise indicated. Where appropriate, the International Nomenclature of Cosmetic Ingredients (INCI) name of ingredients/components is provided. In this regard, the term “(and)” is an INCI designation for a mixture of listed components separated by the term. Any numerical range recited herein is intended to include all sub-ranges subsumed therein, and such ranges are understood to include each and every number and/or fraction between the stated range lower and upper values. Moreover, the term “about” and may refer to deviations of ±20%.

### Anti-Acne Active Ingredient

Skin care compositions may include one or more anti-acne active ingredients to prevent or treat acne. Anti-acne active ingredients may include, without limitation, benzoyl peroxide, salicylic acid, retinoids (e.g., adapalene, tretinoin, etc.), azelaic acid, antibiotics (clindamycin, erythromycin, sulfacetamide, etc.), sulfur ingredients, or combinations thereof. Traditional dosage forms and use levels for anti-acne topical compositions are readily determined (Russell, J. J., Am Fam Physician. 2000 Jan. 15; 61(2):357-365). Preferably, skin care compositions include sulfur ingredients or salicylic acid owing to commercial availability without a prescription and to the efficacy or safety from such anti-acne active ingredients.

Sulfur ingredients may be selected from elemental sulfur (in a variety of forms), sulfides, sulfites, mercaptans, or combinations thereof. Elemental sulfur may include, without limitation, colloidal sulfur, coated sulfur, enrobed sulfur, entrapped sulfur, fumed sulfur, precipitated sulfur, sublimed sulfur, or combinations thereof. Inorganic sulfides may include, without limitation, selenium sulfide, sodium thiosulfate, those with the formula RS, RSH, R₂S, RSSR, RSSH (where R is an inorganic element bound ionically or covalently with sulfur), or combinations thereof. Organic sulfides may include, without limitation, those with the formula RS, R₂S, RSH, R′SSR', R′SSH (where R′ is an organic compound bound ionically or covalently with sulfur), or combinations thereof. Inorganic sulfites may include, without limitation, sulfites, meta bisulfites, or combinations thereof.

While sulfur ingredients may also be selected from sulfates, preferably skin care compositions exclude (are free of) sulfates. Additionally or alternatively, sulfur ingredients may be selected only from elemental sulfur. Thus, for example, the anti-acne active agent in skin care compositions may include precipitated sulfur (e.g., sulfur powder, H₂s, ≥99.5% including 99.999% (metal basis), CAS No: 7704-34-9, about 32.05 g/mol, etc.). In another embodiment the anti-acne active agent may be an ingredient other than a sulfur ingredient, alone or in combination with one or more other anti-acne agents. Thus, for example, skin care compositions may include salicylic acid alone or with one or more other anti-acne agents.

Anti-acne active ingredients may be present from about 0.001 wt. % to about 20.0 wt. %, based on the total weight of the skin care composition. An anti-acne active ingredient may, for example, be present in an amount of 0.0025 wt. %, 0.008 wt. %, 0.1 wt. %, 0.5 wt. %, 1.0 wt. %, 2.5 wt. %, 5.0 wt. %, 7.5 wt. %, 10.0 wt. %, 12.0 wt. %, 15.0 wt. %, 18.0 wt. %, 22.0 wt. %, including every number and/or fraction therebetween, based on the total weight of the skin care composition. For example, sulfur powder may be present in an amount of 5.0 wt. % to 15.0 wt. %, including every number and/or fraction therebetween, such as 10.0 wt. %, based on the total weight of the skin care composition. Additionally or alternatively, salicylic acid may be present in an amount of 0.5 wt. % to 2.0 wt. %, including every number and/or fraction therebetween, based on the total weight of the skin care composition.

### Odor Neutralizer

Skin care compositions may include odor neutralizers to reduce, inhibit, and/or eliminate the malodor of another ingredient present in the skin care composition. Odor neutralizers may be selected from perfumes, metal oxides, mineral powders, polymers (e.g., polymers in salt form), or combinations thereof. Perfumes may include, without limitation, naturally derived fragrances (extracts, essential oils, etc.), synthetically derived fragrances (small molecules), or combinations thereof. Metal oxides may include, without limitation, zinc oxide, titanium oxide, or combinations thereof. Mineral powder may include, without limitation, bentonite, hectorite, smectite, kaolin, saponite, carbonate, or combinations.

Preferably, odor neutralizers are selected from polymers. Care must be taken to minimize ingredients that exacerbate acne formation, irritation, or sensitivity. In one example, the odor neutralizer may be selected from a copolymer of taurides and itaconic acid. Generally, taurides may refer to a group of relatively mild anionic surfactants composed of a hydrophilic head group, such as N-methyltaurine (2-methylaminoethanesulfonic acid), and a lipophilic residue, such as a relatively long-chain carboxylic acid (fatty acid), both linked (e.g., via an amide bond). Useful fatty acids may include, without limitation, lauric (C₁₂), myristic (C₁₄), palmitic (C₁₆) or stearic acid (C₁₈), and combinations thereof (e.g., oleic acid (C_{18:1}), coconut fatty acid (C₈-C₁₈), etc.). Moreover, the polymer may be in salt form. In this regard, one or more counterions may be selected from alkali metals, alkaline earth metals, transition metals, post-transition metals, or combinations thereof. Thus, for example, the odor neutralizer may include a copolymer having at least one zinc counterion. The odor neutralizer may also include, for example, a mixed salt form (e.g., sodium ions and zinc ions, etc.).

While odor neutralizers may be composed of a mixture of ingredients, skin care compositions may exclude (be free of) perfumes, metal oxides, mineral powders, or combinations thereof. In one example, a copolymer in salt form may be the only odor neutralizer present in a skin care composition to reduce, inhibit, and/or eliminate the malodor of a sulfur ingredient. For example, sodium/zinc acryloyldimethyltaurate/itaconate copolymer (the mixed sodium and zinc salt of the copolymer produced by the reaction of 2-acrylamido-2-methylpropane sulfonic acid and itaconic acid) may be the only odor neutralizer present to reduce, inhibit, and/or eliminate the malodor of a sulfur ingredient.

Odor neutralizers may be present in an amount of about 0.001 wt. % to about 5.0 wt. %, based on the total weight of a skin care composition. An odor neutralizer may, for example, be present in an amount of 0.008 wt. %, 0.06 wt. %, 0.1 wt. %, 0.5 wt. %, 0.75 wt. %, 1.0 wt. %, 1.5 wt. %, 2.5 wt. %, 4.0 wt. %, 5.0 wt. %, 6.0 wt. %, including every number and/or fraction therebetween, based on the total weight of the skin care composition. For example, sodium/zinc acryloyldimethyltaurate/itaconate copolymer may be present in an amount of 0.5 wt. % to 1.5 wt. %, based on the total weight of the skin care composition.

### Alpha Hydroxy Acids

Skin care compositions may include one or more alpha hydroxy acids (AHAs). Generally, AHAs are organic carboxylic acids having one hydroxyl group attached directly to the alpha position of the aliphatic or alicyclic carbon atom, but not to a benzene or other aromatic ring. AHAs may include those acids that have additional carboxyl groups. AHAs may be present as alkyl AHAs, aralkyl AHAs, polycarboxyl AHAs, or combinations thereof.

Alkyl AHA side chain radicals attached to the alpha carbon may be hydrogen atoms or simple hydrocarbons called alkyl groups. The generic structure may be represented as (R₁)(R₂)C(OH)COOH, where R₁ and R₂ may be independently H or alkyl group. Alkyl AHAs may exist as stereoisomers as D, L and DL or R, S and RS forms when R₁ and R₂ are not identical. The alkyl groups may be non-aromatic radicals such as methyl, ethyl, propyl, isopropyl, butyl, pentyl, octyl, lauryl and stearyl. Useful alkyl AHAs include, without limitation, 2-hydroxyethanoic acid (glycolic acid), 2-hydroxypropanoic acid (lactic acid), 2-methyl-2-hydroxypropanoic acid (methyllactic acid), 2-hydroxybutanoic acid, 2-hydroxypentanoic acid, 2-hydroxyhexanoic acid, 2-hydroxyheptanoic acid, 2-hydroxyoctanoic acid, 2-hydroxyeicosanoic acid, 2-hydroxytetraeicosanoic acid (cerebronic acid), 2-hydroxytetraeicosenoic acid, or combinations thereof.

Aralkyl AHAs include AHA having aralkyl groups, where aralkyl refers to aryl plus alkyl. Aralkyl AHAs may be formed when a phenyl group or other aromatic ring is attached to the alpha carbon of the alkyl AHA. The generic structure may be represented as (R₁)(R₂)C(OH)COOH, where R₁ and R₂ may be independently H, aryl or aralkyl group. Aralkyl AHAs may exist as stereoisomers as D, L and DL or R, S and RS forms when R₁ and R₂ are not identical. The aryl group may include at least one aromatic radical such as phenyl, diphenyl, biphenyl and naphthyl. The aralkyl group may include at least one aromatic radical and one non-aromatic radical such as a phenylmethyl (benzyl), phenylethyl, phenylpropyl, diphenylmethyl, diphenylethyl, biphenylmethyl and naphthylmethyl group. The hydroxyl group may be attached to the non-aromatic alpha carbon atom. Useful aralkyl AHAs may include, without limitation, 2-henyl-2-hydroxyethanoic acid (mandelic acid), 2,2-diphenyl-2-hydroxyethanoic acid (benzilic acid), 3-phenyl 2-hydroxypropanoic acid (3-phenyllactic acid), 2-phenyl-2-methyl-2-hydroxyethanoic acid (atrolactic acid), or combinations thereof.

Polycarboxy AHAs may include two or more carboxyl groups. The generic structure may be represented as (R₁)(R₂)C(OH)COOH, where R₁ and R₂ may be independently H, COOH, CH2COOH or CHOHCOOH. Polycarboxy AHAs may exist as stereoisomers as D, L and DL or R, S and RS forms when R₁ and R₂ are not identical. Useful polycarboxy AHAs may include, without limitation, 2-hydroxypropane-1,3-dioic acid (tartronic acid), 2-hydroxybutane-1,4-dioic acid (malic acid), 2,3-dihydroxybutane-1,4-dioic acid (tartaric acid), 2-hydroxy-2-carboxypentane-1,5-dioic acid (citric acid), isocitric acid, or combinations thereof.

AHAs may be present in an amount of about 0.1 wt. % to about 10.0 wt. %, based on the total weight of a skin care composition. An AHA may, for example, be present in an amount of 0.08 wt. %, 0.1 wt. %, 0.5 wt. %, 1.0 wt. %, 1.5 wt. %, 1.75 wt. %, 2.0 wt. %, 2.25 wt. %, 2.75 wt. %, 3.5 wt. %, 5.0 wt. %, 7.5 wt. %, 12.0 wt. %, including every number and/or fraction therebetween, based on the total weight of the skin care composition. For example, skin care compositions may include a mixture of at least one alkyl AHA, aralkyl AHA, and polycarboxyl AHA present in an amount about 5.0 wt. % to about 10.0 wt. %, based on the total weight of the skin care composition. Thus, a skin care composition may include a mixture of AHAs consisting of an alkyl AHA selected from the group consisting of lactic acid and glycolic acid, an aralkyl AHA selected from the group consisting of mandelic acid, and a polycarboxy AHA selected from the group consisting of malic acid, tartaric acid, and citric acid, wherein the mixture of the six AHAs may be present in an amount of 4.0 wt. %, 5.0 wt. %, 6.0 wt. %, 7.0 wt. %, 8.0 wt. %, 9.0 wt. %, 10.0 wt. %, 11.0 wt. %, 12.0 wt. %, including every number and/or fraction therebetween, based on the total weight of the skin care composition.

### Other Ingredient(s)

Skin care compositions may include other ingredients such as, without limitation, carriers, sunscreen filters, film formers, surfactants, anti-aging actives, insect repellants, amino acids, colorants, pigments, dyes, opacifiers, pH adjusters (e.g., citric acid, sebacic acid, sodium hydroxide, etc.), preservatives (e.g., phenoxyethanol, etc.), preservative boosters (e.g., ethylhexylglycerine, etc.), chelating agents (e.g., EDTA, tetrasodium glutamate diacetate, etc.), stabilizers, sphingolipids, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anti-acne agents, anesthetics, antiseptics, skin cooling compounds, skin pollution protectants, skin penetration enhancers, exfoliants (e.g., phytic acid, physical exfoliation particles, etc.), staining agents, depigmenting agents, hypopigmenting agents, extracts (fruit, flower, plant, vegetable), vitamins, antioxidants, beta hydroxy acids, retinol and its derivatives, propellants, or combinations thereof.

Other ingredients may be present in an amount of about 0.0001 wt. % to about 99.8 wt. %, based on the total weight of the skin care composition. One or more other ingredients may, for example, be present in an amount of 0.00008 wt. %, 0.008 wt. %, 0.01 wt. %, 0.1 wt. %, 0.25 wt. %, 0.5 wt. %, 1.0 wt. %, 2.5 wt. %, 5.0 wt. %, 10.0 wt. %, 20.0 wt. %, 40.0 wt. %, 60.0 wt. %, 90.0 wt. %, 99.8 wt. %, including every number and/or fraction therebetween, based on the total weight of the skin care composition.

Accordingly, for example, skin care compositions may be composed of one or more carriers including, without limitation, water, vegetable oils, esters (e.g., octyl palmitate, isopropyl myristate, isopropyl palmitate, etc.), ethers (e.g., dicapryl ether, dimethyl isosorbide, etc.), C₂-C₄ alcohol (e.g., ethanol, isopropanol, etc.), fatty alcohols (e.g., cetyl alcohol, stearyl alcohol, behenyl alcohol, etc.), isoparaffins (e.g., isooctane, isododecane, isohexadecane, etc.), silicones (e.g., dimethicones, polysiloxanes, etc.), hydrocarbon oils (e.g., mineral oil, petrolatum, isoeicosane polyisobutene, etc.), polyols (e.g., propylene glycol, glycerin, butylene glycol, pentylene glycol, hexylene glycol, etc.), salicylates, adipates, natural-based micelles (e.g., oleosomes), or combinations thereof.

For example, skin care compositions may be composed of one or more carriers that act as skin-conditioning agents (e.g., emollients, humectants, etc.). Examples of useful skin-conditioning agents include, without limitation, esters (e.g., isopropyl myristate, isopropyl palmitate, 1-decene polymer (hydrogenated), esters of benzoic acid such as lauryl/myristyl benzoate, C₁₂-C₁₅ alkyl benzoate, C₁₆-C₁₇ alkyl benzoate, stearyl benzoate, isostearyl benzoate, behenyl benzoate, ethylhexyl benzoate, butyloctyl benzoate, hexyldecyl benzoate, octyldodecyl benzoate, etc.), polyols (e.g., glycerin, dipropylene glycol, hexylene glycol, 2,3-butanediol, 1,10-decanediol, propylene glycol, 1,2-butanediol, pentylene glycol, ethyl hexanediol, caprylyl glycol, decylene glycol, butylene glycol, propanediol, etc.), natural oils (e.g., nut oil, seed oil, fruit oil, etc.), fatty acids (e.g., lanolin, petrolatum, mineral oil, 10-hydroxydecanoic acid, etc.), triglycerides (caprylic/capric triglyceride, etc.), silicones (cyclomethicone, dimethicone, etc.), salicylates (e.g., butyloctyl salicylate, C₁₂-C₁₅ alkyl salicylate, capryloyl salicylic acid, hexydodecyl salicylate, isocetyl salicylate, isodecyl salicylate, tridecyl salicylate, etc.), adipates (e.g., diisopropyl adipate, etc.) isoparaffins (e.g., isohexadecane, isododecane, etc.), amino acids and derivatives or salts thereof (e.g., sodium pyrrolidone carboxylic acid, etc.), oleosomes (oil bodies), or combinations thereof.

In some aspects of embodiments, skin-conditioning agents may be selected from natural ingredients. For example, a skin-conditioning agent may be selected from Ganoderma lucidum (mushroom) extract, Curcuma longa (tumeric) root extract, Camellia sinensis (green tea) leaf extract, Astragalus membranaceus root extract, hyaluronates (and salts thereof), or combinations thereof. In addition to skin conditioning properties, such ingredients may also have rebuilding and antioxidant properties to improve skin health. For example, mushroom extract may have skin regenerating and wound healing properties. Meanwhile, turmeric, rutin, and green tea may have antioxidant properties. Turmeric may also improve skin radiance and even skin tone. Meanwhile, Astragalus membranaceus may improve skin firmness and smoothness while minimizing skin imperfections, inflammation, and sebum production. Additionally or alternatively, other natural-based skin conditioning agents may include bisabolol, amino acids (e.g., lysine, derivatives of amino acids), aloe (e.g., aloe leaf juice, etc.), or combinations thereof. In addition to skin conditioning, such compounds may also have properties to improve skin health. For example, bisabolol may have anti-inflammatory and soothing properties while lysine may assist in collagen synthesis.

Water may also be intentionally added in skin care compositions at an amount greater than 15.0 wt. % such as, for example, 25.0 wt. %, 45.0 wt. %, 58.0 wt. %, 65.0 wt. %, 75.0 wt. %, including every number and/or fraction therebetween, based on the total weight of a hydrous skin care composition. For example, a water-in-oil (W/O) emulsion may have an aqueous dispersed phase stabilized by an emulsifier and disposed in a hydrophobic continuous phase. Water may, however, be intentionally added in an amount of 15.0 wt. % or less, based on a total weight of the skin care composition, to form substantially anhydrous skin care compositions. Moreover, one or more ingredients may have associated with it absorbed and/or adsorbed water that can be carried into the otherwise substantially anhydrous skin care compositions. For economic, sourcing, and/or formulation reasons, it may be disadvantageous to process hygroscopic materials to make them completely free of water. Thus, substantially anhydrous skin care compositions may include 15.0 wt. % or less, preferably 10.0 wt.% or less and more preferably 5.0 wt.% or less (e.g., ≤1.0 wt. %), of water based on a total weight of the skin care composition.

Carriers may be present from about 0.001 wt. % to about 99.0 wt. %, based on the total weight of the skin care composition. A carrier may, for example, be present in an amount of 0.0008 wt. %, 0.002 wt. %, 0.01 wt. %, 0.1 wt. %, 1.0 wt. %, 5.0 wt. %, 10.0 wt. %, 15.0 wt. %, 18.0 wt. %, 20.0 wt. %, 40.0 wt. %, 60.0 wt. %, 90.0 wt. %, 99.0 wt. %, including every number and/or fraction therebetween, based on the total weight of the skin care composition.

Sunscreen filters are capable of substantially absorbing, scattering, and/or deflecting UV radiation on a keratinous substrate. Preferably, the sun protection factor (SPF) of a sunscreen composition is 8 and the UVA protection factor (UVAPF) is at least ⅓ the SPF. Physical or mineral sunscreen filters may include metal oxides (e.g., titanium dioxide, zinc oxide, combinations thereof, etc.) that may be used in effective amounts to provide an SPF of 8 or more (in contrast to other uses as colorants, etc.). Such filters may have various morphologies such as crystalline form (e.g., rutile, etc.), size (e.g., 5 nm-1.0 μm, etc.), shape (e.g., amorphous, spherical, etc.), and/or functionalization (e.g., coatings, etc.) that allow for the effective use as sunscreen filters. For example, titanium dioxide may have a coating of silicone copolymers, fatty acids, salts of fatty acids, oxides of silicon, oxides of aluminum, oxides of zirconium, or combinations thereof. Organic sunscreen filters may include a p-aminobenzoic acid, a benzophenone, a camphor, a cinnamate, a dibenzoylmethane, a salicylate, an imidazole, a triazole, a triazine, a triazole, or combinations thereof.

Sunscreen filters may, for example, be present in an amount of about 1.0 wt. % to 40.0 wt. %, based on the total weight of the sunscreen composition. For example, one or more sunscreen filters may be present in an amount of 0.8 wt. %, 1.0 wt. %, 5.0 wt. %, 10.0 wt. %, 15.0 wt. %, 20.0 wt. %, 30.0 wt. %, 41.0 wt. %, including every number and/or fraction therebetween, based on the total weight of the sunscreen composition.

Antioxidants may also be added to counter free radicals that damage biomolecules. Antioxidants may include, for example, carotenoids (e.g., beta-carotene, lycopene, lutein, zeaxanthin), polyphenols (e.g., resveratrol, flavonoids such as rutin and catechins, hydroxycinnamic acid, etc.), selenium, vitamins, derivatives thereof, or combinations thereof. For example, a vitamin may be selected from vitamin A (retinoic acid), vitamin B (e.g., nicotinic acid or nicotinamide, B3 niacinamide, etc.), vitamin C (ascorbic acid), vitamin E (tocopherol), or combinations thereof. Meanwhile, a vitamin derivative may be selected from esters of vitamin A (e.g., retinyl acetate, etc.), vitamin C (ascorbyl palmitate, etc.), vitamin E (e.g., tocopheryl acetate, tocopheryl palmitate, tocopheryl linoleate, etc.), or combinations thereof. Additionally or alternatively, antioxidants may include, without limitation, ubiquinone, ferulic acid, resveratrol, extracts from fruit, berries, vegetables, plants, and/or nuts (e.g., grape seed extract, etc.), Coenzyme Q10, or combinations thereof.

Antioxidants may be present from about 0.0001 wt. % to about 1.0 wt. %, based on the total weight of the skin care composition. For example, Camellia sinensis (green tea) extract may be present in an amount of 0.002 wt. %, 0.01 wt. %, 0.045, 0.098, 0.15 wt. %, 0.2 wt. %, 0.3 wt. %, 0.5 wt. %, 1.2 wt. %, including every number and/or fraction therebetween, based on the total weight of the skin care composition.

Film former polymers may be capable of providing a substantially homogenous and/or structured hydro-lipidic film on skin. Generally, film former polymers may be synthesized from two or more different monomers to form copolymers and/or may from individual polymer chains that are connected by bridging molecules or crosslinking agents to form crosslinked copolymers known as cross polymers. For example, film former polymers may include monomers, copolymers, crosspolymers, and/or terpolymers of an organo-silicone hybrid, (meth)acrylate, abietic acid derivative, olefin, silicone, resin, vinyl acetate (VA), vinyl pyrrolidone (VP), maleate, alkyl ester, long chain or short chain carboxylic acid, or combinations thereof. For example, polymers to form films may have a molecular weight (MW) of about 1,500,000 Daltons (DA) or less.

Film former polymers may include crotonic acid/vinyl C₈-C₁₂ isoalkyl esters/VA/bis-vinyldimethicone crosspolymer, acrylate/dimethicone copolymer, VA/butyl maleate/isobornyl acrylate copolymer, acrylates/C₁₂₋₂₂ alkyl methacrylate copolymer, acrylates/octylacrylamide copolymer, methyl dihydroabietate, trimethylsiloxysilicate, C24_C28 alkyldimethylsiloxy trimethylsiloxysilicate, polymethylsilsesquioxane/trimethylsiloxysilicate, polymethylsilsesquioxane, perfluorononyl dimethicone, trifluoropropyldimethylsiloxy/trimethylsiloxy silsesquioxane, PPG-17/isophorone diisocyanate/dimethylol propionic acid copolymer, polyurethane (e.g., 32, 34, 35, 48, etc.), hydrolyzed wheat protein/PVP crosspolymers, maltodextrin/VP copolymer, butylated PVP copolymer, VP/polycarbamylpolyglycol ester, VP/dimethylaminoethylmethacrylate/polycarbamyl polyglycol ester, VP/Dimethiconyl-acrylate/polycarbamyl polyglycol ester, VP/eicosene copolymer, adipic acid/diglycol crosspolymer, trimethylpentanediol/adipic acid/glycerin crosspolymer, polyester-7 (and) neopentyl glycol diheptanoate, polyester-10 (and) propylene glycol dibenzoate, hydrogenated polycyclopentadiene (and) isododecane, stearyl/octyldodecyl citrate crosspolymer, hydroxyethyl acrylate/sodium acryloyldimethyl taurate copolymer, polysilicone-11, or combinations thereof.

Film former polymers may be present in an amount of about 0.1 wt. % to about 5.0 wt. %, based on the total weight of the skin care composition. For example, one or more film former polymers may be present in an amount of 0.08 wt. %, 0.5 wt. %, 1.0 wt. %, 2.0 wt. %, 2.5 wt. %, 3.5 wt. %, 5.1 wt. %, including every number and/or fraction therebetween, based on the total weight of the skin care composition.

Surfactants may include anionic, cationic, nonionic, amphoteric, or combinations thereof. Examples of useful anionic surfactants include, without limitation, carboxylates, amino acid derivatives, alkyl sulphates, alkyl ether sulfates, sulphonates, isethionates, taurates, sulfosuccinates, alkyl sulfoacetates, phosphates, alkyl phosphates, or combinations thereof. Examples of useful cationic surfactants include, without limitation, alkylamines, alkylimidazolines, quaternary ammonium compounds (QAC), or combinations thereof. Examples of useful nonionic surfactants include, without limitation, alkanolamides, amine oxides, esterified carboxylic acids, ethoxylated alcohols, poloxamers, or combinations thereof. Examples of useful amphoteric/zwitterionic surfactants include, without limitation, betaines, sultaines, cocamphoacetates, or combinations thereof.

Examples of emulsifiers, which are surfactants that can stabilize emulsions, may include oil-in-water (O/W) emulsifiers, W/O emulsifiers, or combinations thereof. An O/W emulsifier may have a hydrophilic-lipophilic balance (HLB)≥7, for example 8 to 18. Examples of useful O/W emulsifiers include, without limitation, certain sucrose esters (e.g., sucrose stearate, sucrose laurate, etc.), sorbitan esters (e.g., sorbitan laurate, etc.), polyglyceryl esters (e.g., polyglyceryl-3 methylglucose distearate, etc.), ethoxylated fatty ethers and ethoxylated fatty esters having a HLB≥7, or combinations thereof. Examples of useful natural O/W emulsifiers include, without limitation, cetearyl alcohol (and) cetearyl glucoside, glyceryl stearate (and) cetearyl alcohol (and) sodium stearoyl lactylate, cetearyl wheat straw glycosides (and) cetearyl alcohol, cetearyl olivate (and) sorbitan olivate, or combinations thereof. Thus, a skin care composition may include O/W emulsifiers such as glyceryl stearate, PEG-100 stearate, cetearyl alcohol (and) coco-glucoside, cetearyl alcohol (and) dicetyl phosphate (and) ceteth-10 phosphate, polysorbate 80, tribehenin PEG-20 esters, decyl glucoside, or combinations thereof.

Meanwhile, a W/O emulsifier may have a HLB of <7, for example from 4 to 6. Examples of W/O emulsifiers include, without limitation, certain sucrose esters (e.g., sucrose distearate, etc.), sorbitan esters (e.g., sorbitan oleate, sorbitan dioleate, sorbitan stearate, etc.), polyglyceryl esters (e.g., polyglyceryl-4 isostearate, polyglyceryl dimerate isostearate, etc.), silicones (e.g., a copolymer of a silicone, an alkoxylated derivative of a silicone, or combinations thereof, such as cetyl polyethylene glycol (PEG)/polypropylene glycol (PPG)-10/1 dimethicone, lauryl PEG-8 dimethicone, etc.), fatty acid esters (e.g., glyceryl stearate, etc.), or combinations thereof. Examples of useful natural W/O emulsifiers include cetearyl olivate. Thus, a skin care composition may include W/O emulsifiers such as lauryl PEG-8 dimethicone, cetyl PEG/PPG-10/1 dimethicone, or combinations thereof. It is, however, understood that other emulsion formats may similarly be used such as, without limitation, oil-in-oil (O/O) or oil-in-silicon (O/S) emulsions (e.g., castor oil in silicone oil, etc.) to deliver ingredients (e.g., anti-acne agents, sunscreen filters, antioxidants, etc.) to a substance.

Surfactants may be present from about 0.1 wt. % to about 7.5 wt. %, based on the total weight of the skin care composition. A surfactant may, for example, be present in an amount of 0.008 wt. %, 0.1 wt. %, 1.0 wt. %, 2.0. wt. %, 3.0 wt. %, 4.0 wt. %, 5.0 wt. %, 6/0 wt. %, 7.0 wt. %, 8.0 wt. %, 9.0 wt. %, including every number and/or fraction therebetween, based on the total weight of the skin care composition.

Thickeners may alter viscosity. Thickeners may include plant-based thickeners, mineral thickeners, wax thickeners, ionic thickeners, synthetic polymer thickeners, or combinations thereof. Plant-based polymer thickeners may include, without limitation, xanthan gum, konjac gum, guar gum, acacia gum, acacia senegal gum, cellulose derivatives such as hydroxycellulose, hydroxyethyl cellulose, or combinations thereof. Mineral thickeners may include, without limitation, silica, bentonite, magnesium aluminum silicate, or combinations thereof. Natural or synthetic waxes may include, without limitation, animal waxes, vegetable waxes, mineral waxes, petroleum waxes, silicone waxes, or combinations thereof. Waxes with a melt point from about 50° C. to 100° C. may act as setting agents to solidify a formula. Ionic thickeners may include, without limitation, halide thickeners such as magnesium chloride, sodium chloride, potassium chloride, zinc chloride, or combinations.

Synthetic thickeners may include crosslinked polymers such as alkyl acrylate polymers in which the co-monomers consist of at least one of the following: acrylic acid, sodium acrylate, methacrylic acid, or alkyl acrylate. Polymers consisting purely of acrylic acid are often referred to as carbomers. Thus, for example, single molecules of carbomers can be crosslinked to provide a crosspolymer with a MW over 1 billion DA. In one example, acrylic acid may be crosslinked with allyl sucrose or allyl pentaerythritol. Similarly, a copolymer of C₁₀₋₃₀ alkyl acrylates and one or more monomers of acrylic acid, methacrylic acid or one of their simple esters may be crosslinked (e.g., with an allyl (2-propenyl) ether of sucrose or an allyl ether of pentaerythritol) to form acrylates/C₁₀₋₃₀ alkyl acrylate crosspolymer. Other synthetic thickeners may include acrylate copolymers and crosspolymers such as, without limitation, sodium acrylate/sodium acryloyldimethyl taurate copolymer, polyacrylate crosspolymer-6, or combinations thereof.

Thickeners may be present from about 0.001 wt. % to about 40.0 wt. %, based on the total weight of the skin care composition. A plant-based, mineral, synthetic polymer, or ionic thickener may, for example, be present in an amount of 0.002 wt. %, 0.008 wt. %, 0.01 wt. %, 0.05 wt. %, 0.1 wt. %, 0.5 wt. %, 1.0 wt. %, 2.0 wt. %, 3.0 wt. %, 3.5 wt. %, 4.0 wt. %, including every number and/or fraction therebetween, based on the total weight of the skin care composition. Meanwhile, wax thickeners (solidifying agents) may be present in an amount up to about 40.0 wt. % such as, for example, 1.0 wt. %, 2.0 wt. %, 5.0 wt. %, 18.0 wt. %, 25.0 wt. %, 44.0 wt. %, including every number and/or fraction therebetween.

### Product Format(s)

Skin care compositions may include one or more formulation formats such as, without limitation, an emulsion (O/W, W/O, O/O, O/S, multiple emulsion, etc.), a gel, a lotion, a cream, a spray, a mist, a stick, a mousse, a foam, an ointment, a milk, a base, a concentrate, or combinations thereof. Such formats may initially or finally be substantially aqueous or substantially anhydrous. For example, a substantially anhydrous base may be used in an aqueous emulsion to form a lotion or a cream. Similarly, ingredients and use levels to make such formats may be readily adjusted and mixed in a single phase or in multiple phases to provide a desired commercial sunscreen product. Thus, for example, water content, alcohol content, oil content, emulsifiers, foaming agents, gelling agents, etc., may be included, omitted, adjusted, or mixed in a single phase or in multiple phases to provide a commercial skin care product that accounts for cost, aesthetics, and/or product performance objectives.

Moreover, skin care compositions can be filled into any number of bottles, tottles, tubes, etc., having a variety of closing structures, pumps, sprays (e.g., mists, aerosols, bag on valve), amongst others, or combinations thereof. A bag-on-valve system may, for example, be used to deliver a skin care composition using compressed gas such as air, nitrogen, or carbon dioxide. In addition, skin care compositions may be formulated as a concentrate which, when combined with a propellant, forms a continuous aerosol spray commercial product. Propellants may include, for example, isobutene, butane, propane, dimethyl ether, methyl ether, tetraflouroethane, 1,1-difluoroethane, nitrogen, or combinations thereof. Propellants generally exist as equilibrium of vapor and liquid and can be either dissolved in or miscible with the composition. Ratios of propellant to the concentrate can be adjusted to account for cost, aesthetics, and product performance objectives. Thus, the concentrate and the propellant may be added to an aerosol container in a ratio of, based on weight, about 90:10 to about 60:40, for example about 75:25, about 70:30, and so on. In one example, the aerosol propellant may be present in an amount of about 10 wt. % to about 70 wt. %, based on a total weight of the skin care composition (e.g., final composition).

### Example Anti-Acne Skin Care Compositions

Ingredients of example skin care composition are mixed in phases using traditional techniques to form emulsions. While the example compositions may be implemented independently, the example compositions may be useful as an anti-acne system when two or more are applied (e.g., sequentially). Thus, for example, a cleansing composition may first be applied and rinsed, followed by the application of a treatment composition to clear acne spots and finally by the moisturizing composition which may be kept on the skin over a prolonged period of time (e.g., overnight, etc.). Notably, the anti-acne system may be free of oil, sulfates, alcohol (such as ethanol), parabens, colorants, fragrances, or combinations thereof.

Table 1 shows an example foaming cleansing composition that can be administered from a pump spray by dispensing 1-2 pumps in the hand, applying to damp or dry face, and massaging into the skin in a circular motion. The foaming cleansing composition is rinsed thoroughly with warm water and skin is patted dry. The foaming cleansing composition can be used twice daily, for example in the morning and at night, or as needed. The foam helps to prevent new acne blemishes and blackheads from forming without over drying the skin.

The foaming cleanser composition includes 0.5 wt. % salicylic acid as the anti-acne active ingredient with auxiliary skin benefit ingredients such as turmeric, lactic acid, aloe vera, bisabolol, and green tea extract. The turmeric is a natural antioxidant that helps to gently clarify, nourish, and balance the skin. The lactic acid gently exfoliates for a smoother, more even complexion while boosting skin's moisture levels. The aloe vera provides a soothing adaptogen that helps skin respond to stresses that can trigger an inflammatory response. The bisabolol helps to fight bad bacteria and reduce the appearance of redness and soothe skin. The green tea extract may provide compounds (e.g., polyphenols, etc.) that have antioxidant power or that can penetrate pores to control acne blemishes and blackheads.

EXAMPLE TABLE 1
Foaming Cleansing Composition

| Component (INCI) | Function | wt. % (±20%) |
|---|---|---|
| Example Main Ingredients | | |
| Salicylic Acid | Anti-Acne | 0.5 |
| Bisabolol, Curcuma Longa (Turmeric) Root Extract, | Auxiliary | 0.001-2.0 |
| Aloe Barbadensis Leaf Juice, Camellia Sinensis Leaf | Skin Benefit | |
| Extract | | |
| Alpha Hydroxy Acid (Lactic Acid) | Exfoliator, | 0.5-1.5 |
| | Skin benefit | |
| Example Additional Ingredients | | |
| Water, Butylene Glycol, Glycerin, Propanediol, | Solvent, Skin | Q.S. to 100 |
| Disodium Cocoamphodiacetate, Cocamidopropyl | Conditioner, | |
| Betaine, Cocamidopropyl PG-Dimonium Chloride | Surfactant, | |
| Phosphate, Phenoxyethanol, Ethylhexylglycerin | Preservative | |
| | System | |

Table 2 shows an example treatment composition that can be administered from a tube by applying a thin layer to an affected area one to three times daily, or as needed. If dryness or peeling occurs, the number of applications can be reduced and then increased gradually as tolerated. The treatment composition is relatively gentle and formulated to absorb excess oil, eliminate acne-causing bacteria, and soothe skin.

The treatment composition includes 10 wt. % sulfur as the anti-acne active ingredient with auxiliary skin benefit ingredients such as turmeric, reishi mushroom, lysine, aloe vera, and bisabolol. The active ingredient clears up acne blemishes and blackheads, and allows skin to heal. Meanwhile, the turmeric is a natural antioxidant that helps to gently clarify, nourish, and balance the skin. The reishi mushroom provides healing, detoxifying, and hydrating properties by improving skin integrity while increasing its moisture. The lysine may assist in collagen synthesis. The aloe vera provides a soothing adaptogen that helps skin respond to stresses that can trigger an inflammatory response. The bisabolol helps to fight bad bacteria and reduce the appearance of redness and soothe skin. The composition further includes sodium/zinc acryloyldimethyltaurate/itaconate copolymer to reduce and/or eliminate the odor of the sulfur ingredient. The sulfur-containing treatment skin care composition provides an improved sensory profile (e.g., anti-acne, soothing, healing, and/or minimized odor).

EXAMPLE TABLE 2
Treatment Composition

| Component (INCI) | Function | wt. % (±20%) |
|---|---|---|
| Example Main Ingredients | | |
| Sulfur | Anti-Acne | 10.0 |
| Ganoderma Lucidum (Mushroom) Extract, Bisabolol, | Auxiliary | 0.001-2.0 |
| Curcuma Longa (Turmeric) Root Extract, Aloe Barbadensis | Skin Benefit | |
| Leaf Juice, Camellia Sinensis Leaf Extract | | |
| Sodium/zinc acryloyldimethyltaurate/itaconate copolymer | Odor | 0.1-1.0 |
| | Neutralizer | |
| Example Additional Ingredients | | |
| Water, C₁₂₋₁₅ Alkyl Benzoate, Glycerin, Caprylic/Capric | Solvent, Skin | Q.S. to 100 |
| Triglyceride, Isohexadecane, Pentylene Glycol, Sodium | Conditioner, | |
| Pyrrolidone Carboxylic Acid, 10-Hydroxydecanoic Acid, | Surfactant, | |
| 1,10-Decanediol, Butylene Glycol, Decylene Glycol, | Thickener, pH | |
| Propanediol, Glyceryl Stearate, PEG-100 Stearate, | adjuster, | |
| Polysorbate 80, Magnesium Aluminum Silicate, Acacia | chelating agent, | |
| Senegal Gum, Sodium Acrylate/Sodium Acryloyldimethyl | Preservative | |
| Taurate Copolymer, Xanthan Gum, Salts (zinc chloride, | System | |
| potassium chloride, sodium chloride, magnesium chloride), | | |
| Sodium Hydroxide, Sebacic Acid, Tetrasodium Glutamate | | |
| Diacetate, Phenoxyethanol, Ethylhexylglycerin | | |

Table 3 shows an example moisturizing composition that can be administered from a tube by applying a thin layer to an affected area once daily, for example at night, or as desired. The moisturizing composition is formulated with a blend of AHAs, beta hydroxy acid, and a plant-based acid blend to exfoliate dead skin, to smooth, balance and refine skin texture, and to improve skin brightness and clarity. The moisturizing composition is suitable for acne-prone, combination, oily, and dull or stressed skin. Moreover, the moisturizing composition is relatively gentle while helping to heal and prevent acne blemishes and blackheads.

The moisturizing composition includes 1.0 wt. % salicylic acid as the anti-acne active ingredient with auxiliary skin benefit ingredients such as phytic acid, hydroxycinnamic acid, rutin, turmeric, astragalus, hyaluronic acid, aloe vera, and bisabolol. The active ingredient clears up acne blemishes and blackheads, and allows skin to heal. Meanwhile, the phytic acid is an antioxidant that exfoliates and prevents blackheads or shrinks pores. The turmeric, hydroxycinnamic acid, and rutin are antioxidants that may also help to gently clarify, nourish, and balance the skin. The astragalus balances natural oils found on the skin, reduce imperfections, firms and smooths the skin. The hyaluronic acid replenishes, sooths, and hydrates without clogging pores while providing anti-aging benefits. The aloe vera provides a soothing adaptogen that helps skin respond to stresses that can trigger an inflammatory response. The bisabolol helps to fight bad bacteria and reduce the appearance of redness and soothe skin. The green tea extract may provide compounds (e.g., polyphenols, etc.) that can provide antioxidant power or that can penetrate pores to control acne blemishes and blackheads. Notably, the composition includes six AHAs to exfoliate dead skin and promote cell renewal, to help reduce fine lines, wrinkles, acne and even out skin tone, and to remove dull skin to smooth and help improve the look of skin texture, brightness, and clarity.

EXAMPLE TABLE 3
Moisturizing Composition

| Component (INCI) | Function | wt. % (±20%) |
|---|---|---|
| Example Main Ingredients | | |
| Salicylic Acid | Anti-Acne | 1.0 |
| Phytic Acid, Astragalus Membranaceus Root Extract, | Auxiliary | 0.001-2.0 |
| Ganoderma Lucidum (Mushroom) Extract, Bisabolol, | Skin Benefit | |
| Curcuma Longa (Turmeric) Root Extract, Hydroxycinnamic | | |
| Acid, Rutin, Aloe Barbadensis Leaf Juice, Sodium | | |
| Hyaluronate, Camellia Sinensis Leaf Extract | | |
| Alpha Hydroxy Acid (Lactic Acid, Glycolic Acid, Malic Acid, | Exfoliator, | 5.0-10.0 |
| Mandelic Acid, Tartaric Acid, Citric Acid) | Skin benefit | |
| Example Additional Ingredients | | |
| Water, Dimethicone, Butylene Glycol, Pentylene Glycol, | Solvent, Skin | Q.S. to 100 |
| Glycerin, Propanediol, Caprylic/Capric Triglyceride, | Conditioner, | |
| Dipropylene Glycol, Cetearyl Alcohol, Tribehenin PEG-20 | Surfactant, | |
| Esters, Polyacrylate Crosspolymer-6, Decyl Glucoside, | Thickener, pH | |
| Carbomer, Salts (zinc chloride, potassium chloride, sodium | adjuster, | |
| chloride), Sodium Hydroxide, Lecithin, Polysilicone-11, | Stabilizer, Film | |
| Phenoxyethanol, Ethylhexylglycerin | Former, | |
| | Preservative | |
| | System | |

### ADDITIONAL NOTES AND EXAMPLES

Additional examples include a skin care composition, a consumer-packaged product including a skin care composition, and a method of using or of making a skin care composition.

Example 1 may include a skin care composition comprising an anti-acne active ingredient, and at least one ingredient selected from the group consisting of an odor neutralizer, an alpha hydroxy acid (AHA), or combinations thereof.

Example 2 may include the skin care composition of Example 1, wherein the anti-acne active ingredient comprises benzoyl peroxide, salicylic acid, a retinoid, azelaic acid, an antibiotic, a sulfur ingredient, or combinations thereof.

Example 3 may include the skin care composition of any of Examples 1 to 2, wherein the anti-acne active ingredient comprises salicylic acid, a sulfur ingredient, or combinations thereof.

Example 4 may include the skin care composition of any of Examples 1 to 3, wherein the anti-acne active ingredient comprises a sulfur ingredient including elemental sulfur, a sulfide, a sulfite, a mercaptan, or combinations thereof.

Example 5 may include the skin care composition of any of Examples 1 to 4, wherein the anti-acne active ingredient comprises a sulfur ingredient including colloidal sulfur, coated sulfur, enrobed sulfur, entrapped sulfur, fumed sulfur, precipitated sulfur, sublimed sulfur, or combinations thereof.

Example 6 may include the skin care composition of any of Examples 1 to 5, wherein the anti-acne active ingredient comprises a sulfur ingredient including precipitated sulfur.

Example 7 may include the skin care composition of any of Examples 1 to 6, wherein the anti-acne active ingredient is present in an amount from about 0.001 wt. % to about 20.0 wt. %, based on the total weight of the skin care composition.

Example 8 may include the skin care composition of any of Examples 1 to 7, wherein the anti-acne active ingredient is present in an amount from about 0.5 wt. % to about 10.0 wt. %, based on the total weight of the skin care composition.

Example 9 may include the skin care composition of any of Examples 1 to 8, wherein the odor neutralizer comprises a perfume, a metal oxide, a mineral powder, a polymer or combinations thereof.

Example 10 may include the skin care composition of any of Examples 1 to 9, wherein the odor neutralizer is free of a perfume, a metal oxide, and a mineral powder.

Example 11 may include the skin care composition of any of Examples 1 to 10, wherein the odor neutralizer comprises a polymer in salt form.

Example 12 may include the skin care composition of any of Examples 1 to 11, wherein the odor neutralizer comprises a copolymer having at least one zinc counterion.

Example 13 may include the skin care composition of any of Examples 1 to 12, wherein the odor neutralizer comprises a copolymer in a mixed salt form of sodium ions and zinc ions.

Example 14 may include the skin care composition of any of Examples 1 to 13, wherein the odor neutralizer comprises a copolymer of taurides and itaconic acid.

Example 15 may include the skin care composition of any of Examples 1 to 14, wherein the odor neutralizer comprises sodium/zinc acryloyldimethyltaurate/itaconate copolymer.

Example 16 may include the skin care composition of any of Examples 1 to 15, wherein the odor neutralizer is present in an amount from about 0.001 wt. % to about 5.0 wt. %, based on the total weight of the skin care composition.

Example 17 may include the skin care composition of any of Examples 1 to 16, wherein the odor neutralizer is present in an amount from about 0.5 wt. % to about 1.5 wt. %, based on the total weight of the skin care composition.

Example 18 may include the skin care composition of any of Examples 1 to 17, wherein the AHA comprises an alkyl AHA, an aralkyl AHA, a polycarboxyl AHA, or combinations thereof.

Example 19 may include the skin care composition of any of Examples 1 to 18, wherein the AHA consists of an alkyl AHA, an aralkyl AHA, and a polycarboxyl AHA.

Example 20 may include the skin care composition of any of Examples 1 to 19, wherein the AHA comprises lactic acid, glycolic acid, mandelic acid, malic acid, tartaric acid, and citric acid.

Example 21 may include the skin care composition of any of Examples 1 to 20, wherein the AHA is present in an amount from about 0.1 wt. % to about 10.0 wt. %, based on the total weight of the skin care composition.

Example 22 may include the skin care composition of any of Examples 1 to 21, wherein a mixture of AHAs is present in an amount from about 5.0 wt. % to about 10.0 wt. %, based on the total weight of the skin care composition.

Example 23 may include the skin care composition of any of Examples 1 to 22, further comprising one or more other ingredients including carriers, sunscreen filters, film formers, surfactants, anti-aging actives, insect repellants, amino acids, colorants, pigments, dyes, opacifiers, pH adjusters, preservatives, preservative boosters, chelating agents, stabilizers, sphingolipids, minerals, keratolytics, hormonal compounds, analgesics, anti-allergenic agents, H1 or H2 antihistamines, anti-inflammatory agents, anti-irritants, anti-neoplastics, immune system boosting agents, immune system suppressing agents, anesthetics, antiseptics, skin cooling compounds, skin pollution protectants, skin penetration enhancers, staining agents, depigmenting agents, hypopigmenting agents, extracts, vitamins, antioxidants, beta hydroxy acids, propellants, or combinations thereof.

Example 24 may include the skin care composition of any of Examples 1 to 23, wherein the one or more other ingredients includes at least one auxiliary skin benefit agent.

Example 25 may include the skin care composition of any of Examples 1 to 24, wherein the one or more other ingredients includes at least one auxiliary skin benefit agent comprising Curcuma longa (turmeric) root extract, Ganoderma lucidum (mushroom) extract, Astragalus membranaceus root extract, Aloe barbadensis, Camellia sinensis leaf extract, hydroxycinnamic acid, rutin, sodium hyaluronate, lysine, bisabolol, 10-hycroxydecanoic acid, phytic acid, or combinations thereof.

Example 26 may include the skin care composition of any of Examples 1 to 25, wherein the skin care composition is in the form of an emulsion, a gel, a lotion, a cream, a spray, a mist, a stick, a mousse, a foam, an ointment, a milk, a base, a concentrate, or combinations thereof.

Example 27 may include the skin care composition of any of Examples 1 to 26, further comprising Ganoderma lucidum (mushroom) extract, wherein the anti-acne active ingredient comprises sulfur, and wherein the odor neutralizer comprises sodium/zinc acryloyldimethyltaurate/itaconate copolymer.

Example 28 may include the skin care composition of any of Examples 1 to 26, further comprising Astragalus membranaceus root extract, phytic acid, and hydroxycinnamic acid, wherein the anti-acne active ingredient comprises salicylic acid, and wherein the AHA consists of an alkyl AHA selected from the group consisting of lactic acid and glycolic, an aralkyl AHA selected from the group consisting of mandelic acid, and a polycarboxy AHA selected from the group consisting of malic acid, tartaric acid, and citric acid.

Example 29 may include a consumer-packaged product comprising a container and the skin care composition of any of Examples 1-28 housed in the container.

Example 30 may include a method to provide an anti-acne skin benefit to an individual comprising contacting skin of the individual with the skin care composition of any of Examples 1-28.

Example 31 may include the method of Example 30, wherein the method comprises sequentially contacting the skin with two or more of a cleansing skin care composition, a treatment skin care composition, or a moisturizing skin care composition.

Example 32 may include the method of any of Examples 30 to 31, wherein the anti-acne skin benefit includes relatively less acne on the skin, wherein the method provides an additional benefit comprising one or more of relatively less odor from an ingredient present in the skin care composition or exfoliation of the skin with moisturization, wherein the anti-acne skin benefit and the additional benefit are to be determined relative to a condition of the skin without the application of the skin care composition and/or relative to a condition of the skin with application of a different anti-acne skin care composition.

Example 33 may include a method to make a skin care composition comprising mixing ingredients to form the skin care composition of any of Examples 1-28.

In this disclosure and the following claims, the word “or” indicates an inclusive list such that, for example, the list of X, Y, or Z means X or Y or Z or XY or XZ or YZ or XYZ. Similarly, the list of one or more of X, Y, or Z or a list of X, Y, Z, or combinations thereof means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

While the present disclosure has been particularly described, in conjunction with specific preferred embodiments, it is evident that many alternatives, modifications, and variations will be apparent to those skilled in the art in light of the foregoing description. It is therefore contemplated that the appended claims will embrace any such alternatives, modifications, and variations as falling within the true scope and spirit of the present disclosure.

What is claimed is:

1. A skin care composition comprising:
an anti-acne active ingredient present in an amount from about 0.001 wt. % to about 20.0 wt. %, based on the total weight of the skin care composition, wherein the anti-acne active ingredient comprises salicylic acid, a sulfur ingredient, or combinations thereof; and
an odor neutralizer present in an amount from about 0.001 wt. % to about 5.0 wt. %, based on the total weight of the skin care composition, an alpha hydroxy acid (AHA) present in an amount from about 0.1 wt. % to about 10.0 wt. %, based on the total weight of the skin care composition, or combinations thereof, wherein the odor neutralizer comprises a polymer in salt form, and wherein the AHA comprises an alkyl AHA, an aralkyl AHA, a polycarboxyl AHA, or combinations thereof.

2. The skin care composition of claim 1, wherein the sulfur ingredient comprises elemental sulfur, a sulfide, a sulfite, a mercaptan, or combinations thereof.

3. The skin care composition of claim 2, wherein the elemental sulfur comprises colloidal sulfur, coated sulfur, enrobed sulfur, entrapped sulfur, fumed sulfur, precipitated sulfur, sublimed sulfur, or combinations thereof.

4. The skin care composition of claim 1, wherein the anti-acne active ingredient is present in an amount from about 0.5 wt. % to about 10.0 wt. %, based on the total weight of the skin care composition.

5. The skin care composition of claim 1, wherein the odor neutralizer is free of a perfume, a metal oxide, and a mineral powder.

6. The skin care composition of claim 1, wherein the odor neutralizer is selected from a copolymer having at least one zinc counterion.

7. The skin care composition of claim 1, wherein the odor neutralizer is selected from a copolymer in a mixed salt form of sodium ions and zinc ions.

8. The skin care composition of claim 1, wherein the odor neutralizer includes a copolymer of taurides and itaconic acid.

9. The skin care composition of claim 1, wherein the odor neutralizer includes sodium/zinc acryloyldimethyltaurate/itaconate copolymer.

10. The skin care composition of claim 1, wherein the odor neutralizer is present in an amount from about 0.5 wt. % to about 1.5 wt. %, based on the total weight of the skin care composition.

11. The skin care composition of claim 1, wherein the AHA consists of an alkyl AHA, an aralkyl AHA, and a polycarboxyl AHA.

12. The skin care composition of claim 1, wherein the AHA comprises lactic acid, glycolic acid, mandelic acid, malic acid, tartaric acid, and citric acid.

13. The skin care composition of claim 1, wherein a mixture of AHAs is present in an amount from about 5.0 wt. % to about 10.0 wt. %, based on the total weight of the skin care composition.

14. The skin care composition of claim 1, further including at least one auxiliary skin benefit agent.

15. The skin care composition of claim 14, wherein the at least one auxiliary skin benefit agent comprises Curcuma longa (turmeric) root extract, Ganoderma lucidum (mushroom) extract, Astragalus membranaceus root extract, Aloe barbadensis, Camellia sinensis leaf extract, hydroxycinnamic acid, rutin, sodium hyaluronate, lysine, bisabolol, 10-hycroxydecanoic acid, phytic acid, or combinations thereof.

16. The skin care composition of claim 1, wherein the skin care composition is in the form of an emulsion, a gel, a lotion, a cream, a spray, a mist, a stick, a mousse, a foam, an ointment, a milk, a base, a concentrate, or combinations thereof.

17. The skin care composition of claim 1, further including Ganoderma lucidum (mushroom) extract, wherein the anti-acne active ingredient comprises sulfur, and wherein the odor neutralizer comprises sodium/zinc acryloyldimethyltaurate/itaconate copolymer.

18. The skin care composition of claim 1, further including Astragalus membranaceus root extract, phytic acid, and hydroxycinnamic acid, wherein the anti-acne active ingredient comprises salicylic acid, and wherein the AHA consists of an alkyl AHA selected from the group consisting of lactic acid and glycolic, an aralkyl AHA selected from the group consisting of mandelic acid, and a polycarboxy AHA selected from the group consisting of malic acid, tartaric acid, and citric acid.

19. A consumer-packaged product comprising a container and the skin care composition of claim 1 housed in the container.

20. A method to provide an anti-acne skin benefit to an individual comprising contacting skin of the individual with the skin care composition of claim 1.
